(19) United States
(12) Patent Application Publication
(10) Pub. No.: US 2013/0338464 A1
(43) Pub. Date: Dec. 19, 2013

(54) SELF-CONTAINED BLOOD GLUCOSE TESTING APPARATUS

(71) Applicant: DENNIS Ryan STAINKEN, Hillsborough, NJ (US)

(72) Inventor: DENNIS Ryan STAINKEN, Hillsborough, NJ (US)

(21) Appl. No.: 13/527,448

(22) Filed: Jun. 19, 2012

(51) Int. Cl. A61B 5/151; A61B 5/145

(52) U.S. Cl. 600/365

(57) ABSTRACT

A compact, self-contained apparatus for blood glucose testing, which comprises a blood glucose meter (32), a lancet device (30), and an enclosure for test media with separate compartments for new and used disposable test media (28).

### FEDERALLY SPONSORED RESEARCH

Not Applicable

### SEQUENCE LISTING OR PROGRAM

Not Applicable

REFERENCES CITED
U.S. Patent Documents

| | | |
|---|---|---|
| 4,627,445 | Dec. 9, 1986 | Garcia et al. |
| 4,648,408 | Mar. 10, 1987 | Hutcheson et al. |
| 4,787,398 | Nov. 29, 1988 | Garcia et al. |
| 5,911,937 | Jun. 15, 1999 | Hekal |
| 6,093,156 | Jul. 25, 2000 | Cunningham et al. |
| 6,183,489 | Feb. 6, 2001 | Douglas et al. |
| 6,849,052 | Feb. 1, 2005 | Uchigaki et al. |
| 20020052618 | May 2, 2002 | Haar, Hans-Peter et al. |
| 20040127818 | Jul. 1, 2004 | Roe, Steven N. et al. |
| 20040127929 | Jul. 1, 2004 | Roe, Steven N. et al. |
| 20040215224 | Oct. 28, 2004 | Sakata, Tetsuya et al. |
| 20050013731 | Jan. 20, 2005 | Burke, David W. et al. |
| 20050016844 | Jun. 18, 2004 | Burke, David W. et al. |
| 20050288698 | Dec. 29, 2005 | Matsumoto, Daisuke |
| 20060100543 | May 11, 2006 | Raney; Charles C. et al. |

Foreign Patent Documents

| | | |
|---|---|---|
| WO/2010/021760 | 25.02.2010 | BAYER HEALTHCARE |
| | | LLC |
| WO/2007/086843 | 02.08.2007 | NOVA BIOMEDICAL |
| | | CORPORATION |
| WO/2005/102154 | 03.11.2005 | BECTON, DICKINSON |
| | | AND COMPANY |
| WO/2003/015627 | 27.02.2003 | INVERNESS MEDICAL |
| | | LIMITED |
| WO2001064105 | 03.02.2000 | INVERNESS MEDICAL |
| | | TECHNOLOGY |

### FIELD OF THE INVENTION

An apparatus that combines essential blood glucose testing equipment into a discreet self-contained unit, in order to provide a transportable solution that simplifies the testing procedure and daily use for a patient.

### BACKGROUND OF THE INVENTION

The invention is based on widely available blood glucose test meters and the method of use for determining the blood glucose concentration with single-use disposable test strips adapted for conducting diverse real-time or near real-time assays of analytes.

The invention further relates to a lancet device that arms and releases a lancet needle used to puncture a user's skin and produce a small blood sample for immediate testing.

Diabetics rely heavily on these machines and technology to monitor and control blood glucose levels by receiving information that indicates the need for medication or treatment. As a result, a diabetic patient must regularly test his or her blood sugar multiple times per day in order to make informed treatment decisions and remain healthy.

There are several components needed to perform this blood test routine, which make the transport of all individual components exceedingly difficult for frequent and immediate access. The only devices currently available to patients combine all individual components into a Zippered pouch or test kit, resulting in a bulky cumbersome package that cannot be easily stored or transported in an individual's clothing pocket. Inventions that have tried to address this issue such as WO2005102154 or WO2001064105 have combined a test meter, lancet device, and test strip storage, but have still not created an easily transportable alternative that considers all the needs for on-the-go blood glucose testing by a diabetic patient.

In addition, those inventions have also ignored the need for a solution that is inconspicuous. Those inventions have created a pen-like device and configuration for the combination of testing equipment, perhaps focusing on ease of use for a care-provider rather than the needs of the patient. In public settings, the foreignness of the obtrusive pen like devices or the common, bulky test kit draw unnecessary attention to the user who desires to perform his or her blood glucose test discreetly and without explanation to others. Protecting the delicate psychological nature of dealing with an incurable disease, such as Type I Juvenile Diabetes, is improved when the equipment needed to manage the condition resemble objects commonly found amongst all people, such as a rectangular cell phones or other portable electronic devices. This situation is especially relevant for teenagers and young adults who deal with increased social pressure.

The currently available inventions have failed to include a storage area to hold contaminated disposable test media, which is consistently created after each blood test is completed. Used test media cannot be stored with clean unused test strips. There are endless situations when a public waste receptacle is not available, especially a waste receptacle appropriate for receiving potential biohazard waste that contains human blood. In order to provide a truly transportable, self-contained unit, that allows blood glucose testing in any situation, a designated storage compartment must be available to receive dirty test strips. The user can later find a convenient waste receptacle to empty the dirty compartment when it becomes full.

Another component that is not currently available is one that addresses the additional need of diabetic patients to carry glucose tablets in order to remedy the event of emergency hypoglycemic situations. In these situations diabetics must treat their low blood sugar by administering fast acting sources of concentrated high glucose, preferably manufactured and widely available glucose tablets, which offer a fast acting form of glucose provided in a uniform measurable dose. Failure to treat a low blood sugar quickly and efficiently can create a life threatening situation where a diabetic's brain becomes starved for glucose and partially or completely shuts down, leading to cognitive impairment, dangerous irrational behavior, coma, and death.

Detecting low blood sugar can occur naturally for some who feel physical symptoms such as shakiness, headaches, dizziness, or blurred vision; however, for the majority of diabetic patients, the only reliable method for identifying these dangerous events is by utilizing a blood glucose test meter to routinely test blood sugar levels. This is especially true for patients that have had diabetes for several years and no longer experience physical symptoms.

When a low blood sugar level is detected, the immediate next step for a diabetic is to locate and orally administer glucose tablets. A diabetic is advised to never be without a treatment for low blood sugar. They are also advised to check their blood sugar before all serious engagements, such as operating a motor vehicle, so that they will not unexpectedly be caught in a low blood sugar episode producing irrational behavior that could potentially endanger others.

For all these reasons and more, a superior solution to test blood glucose levels in any location will also combine a way to treat low blood sugars, eliminating the additional need of diabetic patients to carry blood glucose tablets separately.

A similar situation can occur in reverse, when a diabetic experiences hyperglycemia, or high blood sugar, and requires indication from a blood glucose test meter followed by administration of an appropriate treatment method. Fortunately, these events are generally less urgent than hypoglycemia, however they can become emergency situations if left unchecked or untreated. A common form of treatment for hyperglycemia is injection of insulin, which requires the equipment of a vial of insulin and a syringe. Another form of treatment is an orally administered medication. A third form involves the use of an insulin pump with which the user can program an insulin dose and have the pump perform the delivery mechanically. A fourth form is using an insulin pen, which allows easy transport of insulin and quick administration.

For patients that do not use an insulin pump or insulin pen, carrying necessary insulin supplies or pills is an additional burden that can be addressed by the invention that combines the ability to carry these supplies with the blood glucose testing apparatus.

### SUMMARY OF THE INVENTION

The present invention eliminates the bulk, inconvenience, and discomfort of currently available devices by combining the necessary components to store new test strips, draw blood samples, perform analyte analysis, and receive the contaminated test medium into a separate compartment for later disposal. This single compact device is designed to mimic other common handheld devices such as a cellular phone, and can be easily manipulated in a manner that follows a normal testing routine, adding to a diabetic's flexibility in lifestyle, travel, convenience, and comfort.

One object of the invention is to house the meter unit, an electronic component that conducts the sample analysis. This object receives user input, disposable test media, a battery to supply power, and displays processed data results.

Another object embodies a mechanism that receives a disposable lancet and can be moved between a set or ready position and a freed or release position. This action is controlled by a firing button, armed using a sliding access point, and generated by spring power. A removable cap allows the lancet to be replaced and protects the lancet during transport from accidents and contamination when not in use.

Another object consists of two air-tight enclosures, which store used and unused disposable test media and or other material necessary for operation such as disposable lancets, alcohol prep swabs, and a backup battery.

Another object is to provide an enclosure for housing hypoglycemia and or hyperglycemia treatment medication that is recommended to be carried by diabetics at all times in order to treat emergency situations.

### BRIEF DESCRIPTION OF THE DRAWINGS

The above and other objects and advantages will become more apparent upon consideration of the following drawings and detailed description, in which:

FIG. 1A is a front perspective view of an embodiment of the present invention with a meter unit positioned parallel and adjacent to a lancet device.

FIG. 1B is a rear perspective view of an embodiment of the present invention with a meter unit positioned parallel and adjacent to a lancet device.

FIG. 2 is front perspective of an embodiment of the present invention with a meter unit positioned parallel and adjacent to a lancet device.

FIG. 3A is a front view of an embodiment of the present invention with a meter unit positioned parallel and adjacent to a lancet device in operation.

FIG. 3B is front view of an embodiment of the present invention with a meter unit positioned parallel and adjacent to a lancet device with hidden lines visible.

FIG. 4A is a perspective view of an enclosure for housing new test media separately from soiled test media with one section lid open to forty-five degrees, illustrating an exemplary manner of use.

FIG. 4B is a side view of an enclosure for housing new test media separately from soiled test media.

FIG. 5A is a perspective view of an alternative embodiment of the present invention, showing a detachable cartridge enclosure for housing new test media separately from soiled test media.

FIG. 5B is a rear perspective view of an alternative embodiment, showing a detachable cartridge enclosure for housing new test media separately from soiled test media.

FIG. 6A is a perspective view of an alternative embodiment of the present invention with the meter unit positioned on top of the lancet device, illustrating an exemplary manner of use for a sliding cover.

FIG. 6B is a rear perspective view of an alternative embodiment of the present invention with the meter unit positioned on top of the lancet device.

FIG. 7A is a perspective view of an alternative embodiment of the present invention with an enclosure for housing hypoglycemia treatment medication comprising a horizontal area.

FIG. 7B is a perspective view of an alternative embodiment of the present invention with an enclosure for housing hypoglycemia treatment medication comprising a vertical column area

FIG. 8A is a perspective view of the embodiment of the present invention with a jacket, in an open state, surrounding the outside of the apparatus.

FIG. 8B is a perspective view of the embodiment of the present invention with a jacket, in a closed state, surrounding the outside of the apparatus.

DRAWINGS - Reference Numerals

| | |
|---|---|
| 6 | front of body |
| 8 | back of body |
| 10 | lids of test media enclosure |
| 12 | opening for test media |
| 14 | notch for post |
| 15 | post and groove for top |
| 16 | opening in lancet device top |
| 18 | detachable cover for lancet device |
| 20 | firing button for lancet device |
| 22 | button to arm lancet device |
| 24 | buttons to control meter |
| 26 | display window |
| 28 | enclosure for test media |
| 30 | lancet device |
| 32 | test meter |
| 40 | notch lock on lid |
| 42 | hollow storage area |
| 44 | hinge on lid of test media enclosure |
| 46 | recessed groove on lid |
| 48 | notched locking mechanism |
| 50 | sliding detachable cover |
| 52 | groove for unlocking top |
| 54 | front medication enclosure cover |
| 56 | tab for assisting opening |
| 58 | bottom medication enclosure cover |
| 80 | apparatus jacket |
| 110 | lancet |
| 112 | test strip |
| 114 | glucose tablet |

### DETAILED DESCRIPTION OF THE INVENTION

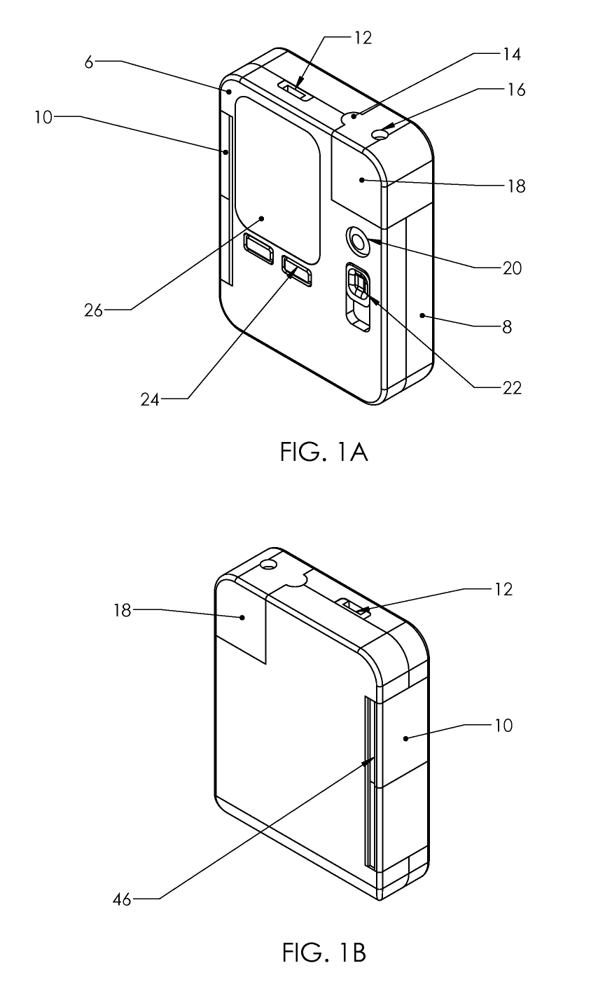

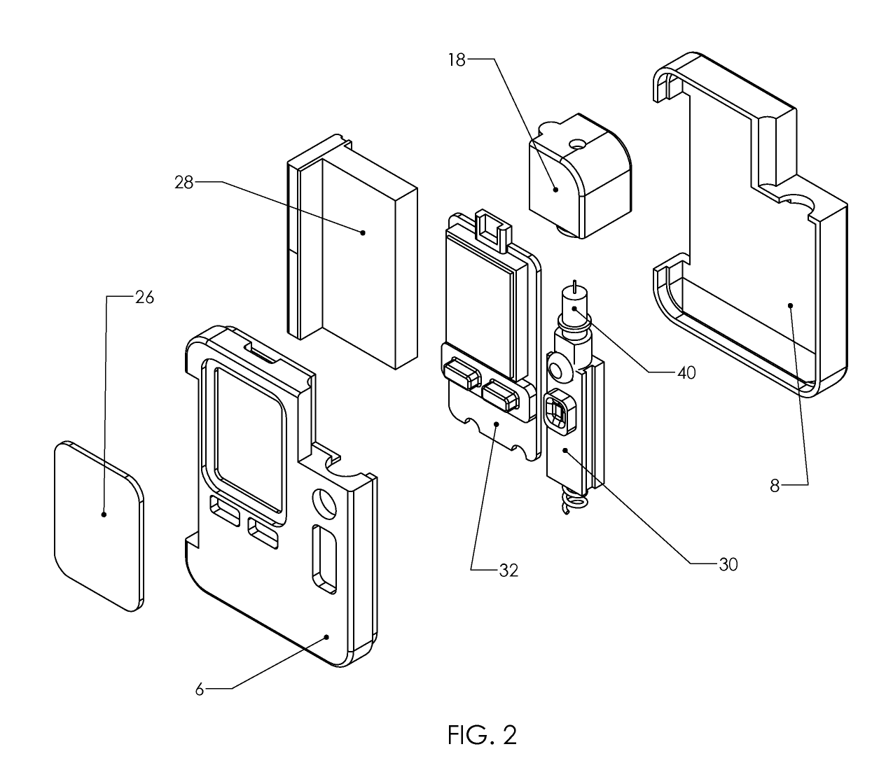

One embodiment of the present invention is illustrated in FIG. 1A (front perspective view) and FIG. 1B (rear perspective view). The unit can be constructed of any suitable material, but is preferably constructed of an engineering plastic material. The front of the body of the apparatus 6 is designed with an outside ridge that overlaps with the back of the body 8 to allow for easy construction and a smooth outside appearance. The design of these two pieces incorporate several openings (in clockwise order around the apparatus): opening for test medium 12 to be inserted, opening for firing button of lancet device 20, opening for sliding button to arm lancet device 22, opening for buttons to control meter 24, an opening for screen to cover meter LCD display 26, and an opening for a battery, not shown. The corners and edges of the base are designed with fillets to allow it to be comfortably held by a user's hand and to be inserted and removed from a user's pocket with ease.

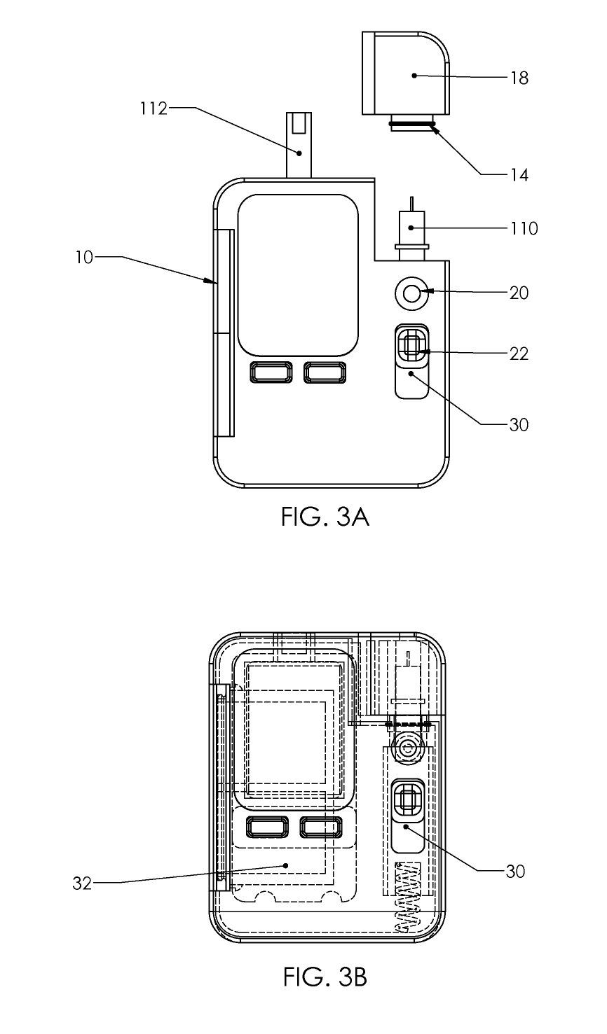

At the top right of the apparatus, a detachable cover for the lancet device 18 is designed with a notch 14 and a post 15 that help to hold the top in place. To remove the cover, a user pulls the cover upwards as shown in FIG. 3A. A user may then remove or replace the lancet 110 that fits concentrically in the lancet body below. An opening in the lancet device top 16 allows for the lancet to protrude from the device, when the lancet device is armed and fired, so that it may puncture the user's skin and produce a blood sample.

Another object of the present invention is a precision depth control lancing tip, such as that described in U.S. Patent Application 20040127818, which is adjustable to control the amount of protrusion of the lancet needle beyond the lancet device top opening. The purpose of this adjustment is to control how large a blood sample is produced on a patient's skin surface based on how far the needle penetrates the outer skin membrane. Another purpose is to provide more comfortable testing because increased levels of discomfort or pain are associated with deeper levels of puncturing by the lancet needle.

Another object of the present invention is the enclosure for housing new test media separately from soiled test media. The present invention has a hollow storage space 42 that has been divided into two spaces by a wall that connects to the top of the lid of the test media enclosure 10 so that contaminates are prevented from entering the space. The storage space comprises two areas, one that is to be used to store new test media, and the other to be used to store soiled or used test media. The spaces can also be used to store other materials chosen by the patient, such as a spare battery for the meter unit, spare lancets, alcohol prep swabs, or other medication.

The enclosure is a self-contained space with walls on all sides, because the storage for unused test strips must be air-tight and possibly accommodate a humidity removing lining such as that described in U.S. Pat. No. 5,911,937. Some test strips that are exposed to air or a non-humidity controlled environment will be damaged, unusable, or unreliable, producing erroneous results if used to conduct blood glucose testing.

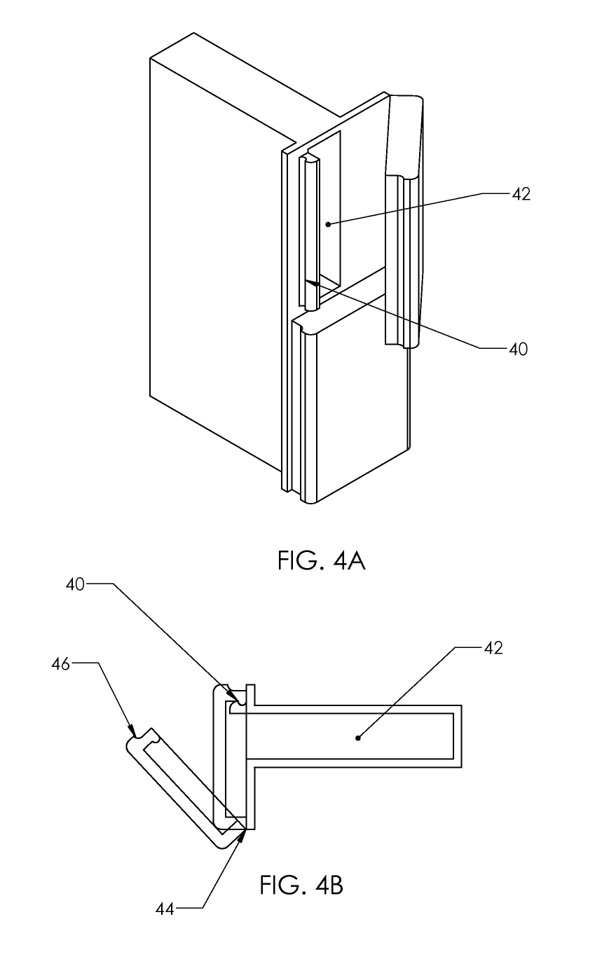

FIG. 4 shows additional details of a test media enclosure with a hinge on the lid of the test media enclosure 44 constructed from any suitable material, preferably an engineering plastic material, and a recessed groove on the lid 46 to allow for the user to open the storage compartment and insert/remove test strips. The notch lock on the lid 40 is just one example of a component to hold the lid in the closed position and maintain air-tightness.

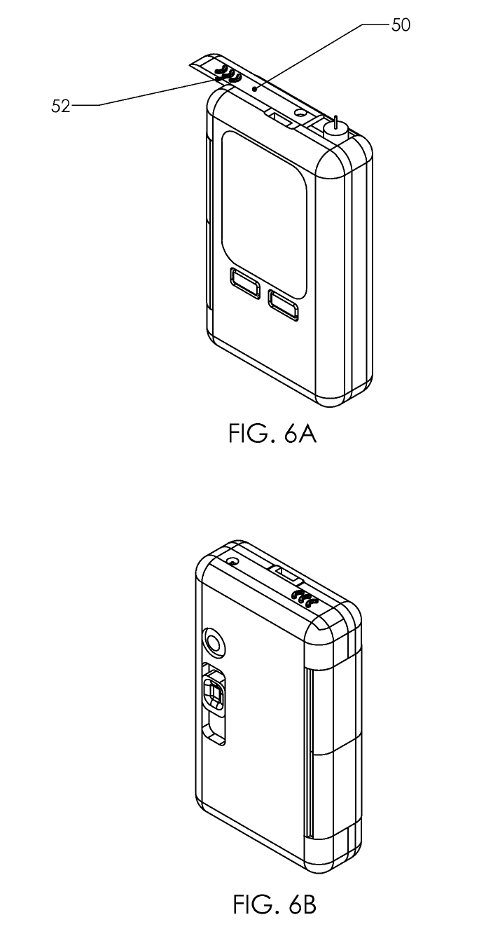

A hinge on the lid of the test media enclosure 44 is only necessary to assist the lid from being disconnected and misplaced by the user. Another embodiment has no hinge and a removable lid. Another embodiment allows the lid to slide off the surface of the storage space, in a fashion similar to that of the sliding detachable lancet cover 50 shown in an alternative embodiment of the apparatus FIG. 6A. In the embodiment, a locking mechanism is created by a lid covering the entire side of the body of the apparatus, fitting into grooves, and sliding parallel to the device in a manner than can used to produce access to the container contents below. The lid can also be created to slide in any direction or to provide access to only one compartment of the enclosure for housing test media at a time.

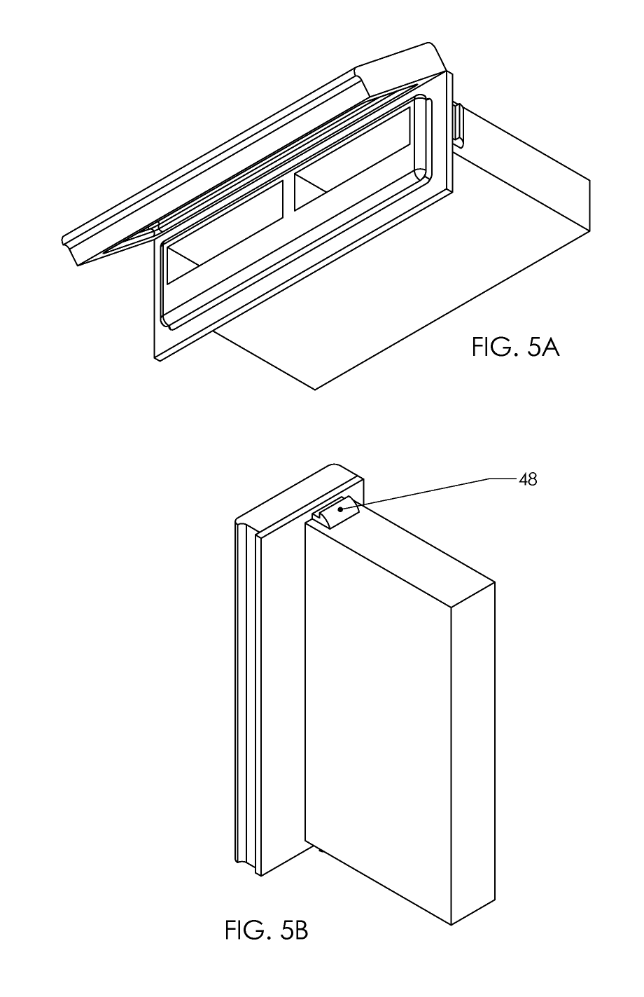

The lids used to cover the test media enclosure can comprise two lids to cover each section as shown in FIG. 4A or one lid to cover both sections as shown in FIG. 5A. Preference is given to one lid in order to simplify manufacturing.

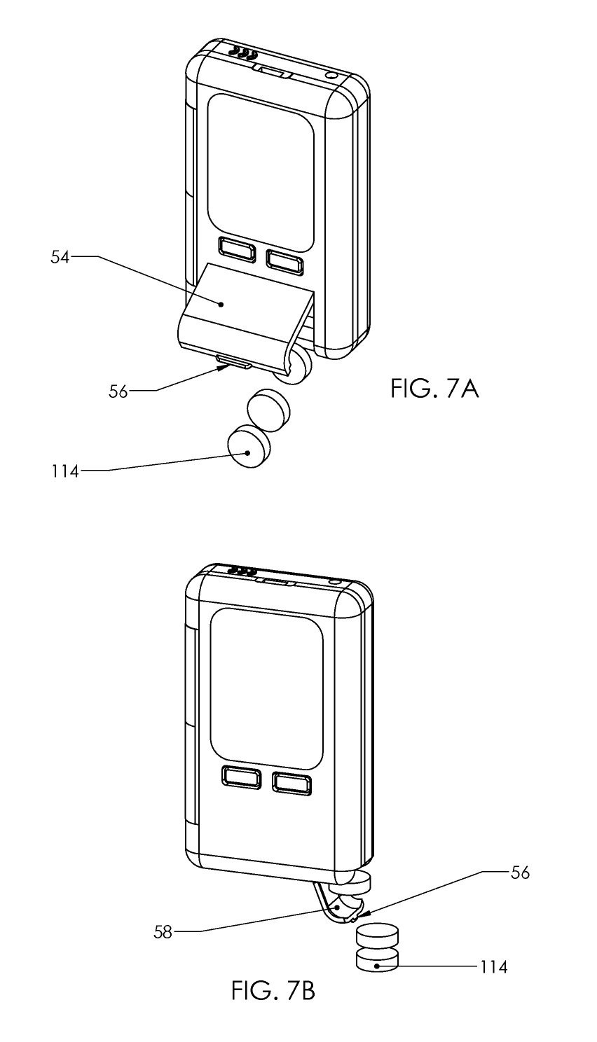

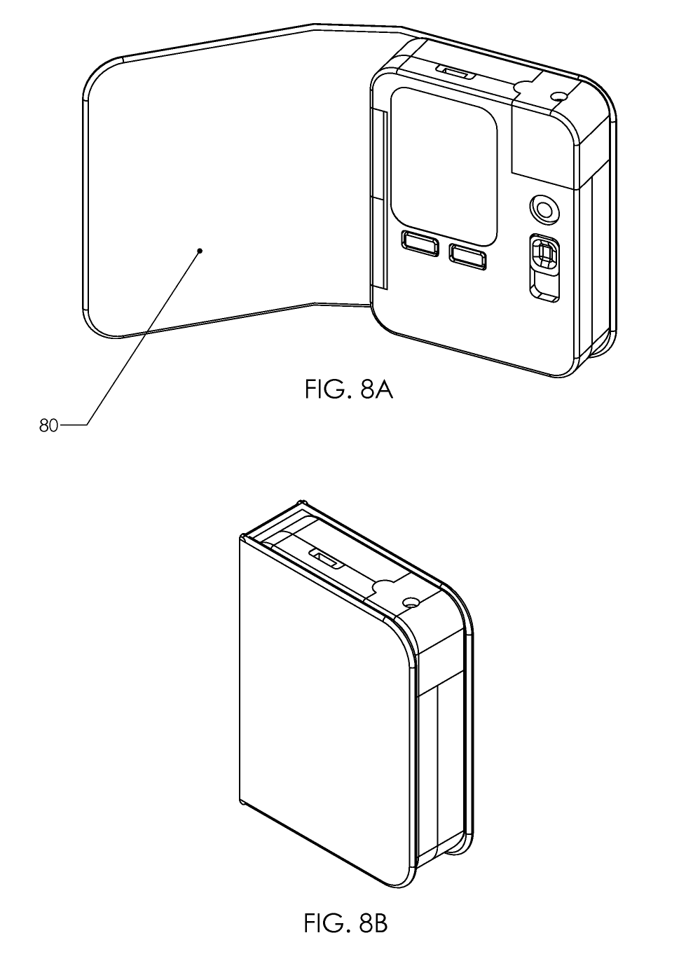

The recessed groove on the lid 46 is given preference for enabling the lid to be disengaged and opened, but is just one embodiment. There are several recessed area configurations such as a circular indentation on the lid with grooves that allow a user's finger to apply force and open the enclosure. Another embodiment is to use an extended tab like that shown in the tab for assisting opening 56 of the enclosure for housing hypoglycemia treatment medication shown in the alternative embodiment of the apparatus in FIG. 7A.

There are many possibilities and arrangements to keep the container as airtight and free from contamination as possible. An alternative embodiment is shown in FIG. 5A, which has protruding walls on all four sides of the lid. Each protruding wall has a notch that overlaps with the lid's notch to keep the enclosure closed. This overlapping notched lock design can be seen in FIG. 4B. The alternative design in 5A includes rounded corners to allow for the lid to close more smoothly and to prevent weak points from developing at corner wall intersections. Another embodiment is a design that utilizes an elliptical locking opening in the lid that fits into elliptically arranged protruding walls. The lid and wall arrangement can also be reversed wherein the lid has a protrusion that fits inside of the walls of the storage space.

The goal for the test media enclosure is to optimize the area for storage of test media and to be robust for withstanding repeated use. The design presented in FIG. 5A and 5B shows the enclosure as a detachable cartridge with a notched locking mechanism 48. The purpose of the test media enclosure being detachable is to overcome design and material robustness concerns by providing the enclosure as replaceable. Another purpose is to replace the test media enclosure if the hollow storage space becomes dirty from use. Another purpose is to allow new test media to be distributed to patients in a convenient container.

The notched locking mechanism 48 is just one possible method for securing the cartridge between the front and back surfaces of the apparatus body. The notches are located on the side of the cartridge and snap the cartridge into position for operation. The cartridge can be removed by forcibly pulling the cartridge outwards away from the apparatus body. The outside surface of the cartridge can be altered with different materials, such as a foam or adhesive, in order to create additional positive friction that can be used to hold the cartridge in place. Another embodiment is a locking mechanism that attaches to the bottom of the cartridge body and locks into a receptacle inside the apparatus.

There are many possibilities and methods for the present invention to embody the position and attachment of the enclosure for housing test media and to allow the lid opening to lock and create an air-tight seal. In one embodiment, the storage area is located completely on the rear of the apparatus, instead of between the top and bottom base layers, with lids for accessing the interior of the enclosure that can be positioned on any side. This storage area is added completely onto the rear of the device in a new space. In another embodiment a similar storage space is removable and attaches by snapping onto the back of the apparatus, meeting the above mentioned goals of an enclosure for housing test media that is detachable. In another embodiment, a lid opens a portion or the entire rear of the apparatus, uncovering a storage area that is created from the free space available around the main device components. In another embodiment, this free space storage area is accessed via the front, bottom, or top of the device.

The direction for opening the lid of the test media enclosure may be constructed in either a rear opening configuration, as demonstrated in FIG. 4B, or a forward opening configuration, as shown in FIG. 5A. Preference is given to a forward opening configuration in order to allow the lid to serve as a platter and prevent the compartment contents from falling out. With the meter LCD display facing the user, the rear opening configuration assists opening by a patient's forefingers, while a forward opening configuration assists opening by a patient's thumb, for right-handed patients.

Another feature of the enclosure for housing test media is a size appropriate to the height of a test strip in order to eliminate excessive space that contributes to rattling of test strip contents and noisy transportation. The hollow storage space 42 in the present invention is only slightly deeper than the height of test media. The width of the compartment opening is also of a specific size as to allow the test media only to be stored vertically within the enclosure, preventing test strips from falling into a horizontal position is difficult for the patient to retrieve or extract strips from the bottom of the compartment. For an idea of the shape and size of test medium relative to the present invention, refer to test strip 112 shown in FIG. 3A.

An alternative embodiment to apparatus of the present invention has a larger, general purpose storage compartment that can be used to hold additional spare lancet needles or individually packaged alcohol prep swabs, used to clean and disinfect a user's skin at the site a user wishes to use for producing a blood sample.

The blood glucose meter receives a test strip 112 via the opening for test media 12. The blood glucose meter further comprises a display window 26, such as an LCD display or like device, which can display any number of test results. A plurality of blood glucose meter operation buttons 24 or controls can be provided to allow a user to control the meter and meter display window. A data connector can be provided with the blood glucose meter 32 for communication access, such as to upload data from other devices or to download data from the device to other devices. The data connector can be disposed on the bottom of the apparatus body and can comprise any number of hardwired or wireless communication connectors.

The blood glucose meter also includes a memory for storing measured blood glucose values, exercises and meals, along with other related data such as corresponding dates, time of day, and duration of each, and the units that were used as these values and events were measured. The meter can also host software that can be programmed to offer the user advice and alerts according to blood glucose patterns and trends.

In order to use the apparatus of the present invention, the patient must first remove the lancet device top 18 by pulling it straight away from the body of the apparatus. Next, the patient inserts a lancet 110 into the lancet device by placing it concentrically into the opening of the body of the lancet device and placing it forcibly downwards. Next, the patient replaces the lancet device top and removes a test strip by using the recessed groove on the storage container lid to open the storage container. The patient then takes the test strip 112 and inserts it into the opening for test media 12 at the top of the unit.

The device should then be rotated onto its side, so that it is perpendicular to the patient's finger from which the blood sample will be drawn. For example, the apparatus should be held with the left hand while it is placed against the pad of the patient's right hand index finger. Next, the button to arm the lancet device 22 is slid backwards until the lancet device locks behind the firing button for the lancet device 20. The firing button can then be pressed with a thumb or free finger, causing the lancet to project rapidly forward, penetrating the opening in the lancet device top 16 and the user's finger. The user can then reposition their finger on the adjacent test medium, transferring the fresh blood sample. When the meter LCD screen displays the results for the test, the test medium can be removed from the test meter and placed into the test media enclosure for soiled test media.

When the enclosure for new test strips becomes empty it can be refilled and when the enclosure for soiled test strips becomes full it can be emptied into an appropriate waste receptacle. The present size of the enclosure for housing test media of the present invention allows for approximately 30-40 test strips to be stored in each of the two compartments. This supply can last a diabetic who tests 5 times a day for approximately one week's duration. Lancet needles can typically last a long time and users will tend to replace these based on their personal preference or when the lancet becomes painful to use, indicating the needle has dulled. The common durations for replacing lancet needles can range from every 2 days to every 2 months to every 21 tests.

Placing used test strips in the used test strips compartment of the enclosure for housing test media will over time cause the compartment to become dirty from dried human blood. While this does not cause damage to the operation of the present invention, the compartment can be cleaned and sanitized.

Another embodiment to aid in cleaning of the used test strip compartment uses a smooth contoured design, without edges, for the bottom of the storage space of the enclosure for housing test media.

An insertable disposable plastic lining, film, or container that fits within the storage space of the enclosure for housing test media is another embodiment of the present invention that can be used to protect the enclosure if it is not replaceable. It can also be used as a method to distribute and replace new test strips into the new test strip compartment.

There are various possibilities for attaching and combining the lancet device with the main meter and storage compartment while still maintaining a discrete shape to the apparatus. Having the lancet device be detachable from the body of the apparatus in the present invention also has advantages in allowing the patient greater access to testing sites, potentially on arm, palm, and thigh, although testing on finger tips is preferably for producing the most accurate and current blood glucose measurements.

In another embodiment, the apparatus of the present invention allows the lancet device to detach from the test meter and test media enclosure. In the embodiment, the rectangular shaped lancet device uses a similar style notch as used for the lancet device top 14, which continues along the side of the entire lancet device assembly. This allows the lancet device to slide parallel against the meter until the two become separated. When the two are in the attached position, the outside appearance of the device appears smooth and unbroken.

In another embodiment the apparatus of the present invention has a pivotable center point, connecting the lancet device and the main meter body and allowing the lancet device to spin 360 degrees around the center pivot. This allows for increased freedom in positioning the lancet device to access testing sites without the test strip interfering. This pivot point can be placed at any position on the lancet device body and can also allow the lancet device to become fully detached via the pivot point.

FIG. 6A and 6B present an alternative embodiment in which the lancet device is constructed beneath the meter and accessed from the back of the apparatus. This design creates a simpler configuration and appearance for the front of the invention that increases the ability of the device to resemble other common portable electronic devices. Concern for this arrangement is given to creating an apparatus that is too thick. The opening used to insert the lancet into the lancet body is located beneath a sliding detachable cover 50. In the embodiment, the lancet device is operated by controls positioned on the back of the apparatus.

Another embodiment of the apparatus of the present invention has the ability to carry a patient's medication to treat hypoglycemia, often in the form of glucose tablets. Diabetics, particularly type I diabetics, must carry glucose tablets or some form of treatment for hypoglycemia, at all times. If a diabetic does not treat hypoglycemia quickly and efficiently he or she risks substantial harm and possibly death. Other effective methods for treating hypoglycemia include: injection of glucagon, high glucose gels or pastes, and ingestion of high glucose containing food such as soft drinks or fruit juice. Preference is given to the use of glucose tablets because they are compact, easily transportable, and affordable.

Current methods for portably and compactly carrying glucose tablets are few as glucose tablets are typically distributed in containers holding large quantities. Preferred transportable methods are individually wrapped glucose tablets or a refillable small plastic tube that can carry stacked glucose tablets, having a detachable cover on one end.

FIGS. 7A and 7B present additional embodiments of the apparatus of the present invention, having an enclosure with a designated storage space for housing glucose tablets or other medication for the treatment of hypoglycemia. In FIG. 7A, the medication is stored vertically in a column like fashion with a hinged lid at the opening in the bottom of the apparatus for removing the medication. The vertical column space can also house a tube of glucose gel or paste medication and a glucagon syringe kit. In FIG. 7B the storage space for the medication comprises a horizontal area at the bottom of the apparatus.

In both cases, the housing is air-tight similar to the enclosure for housing test media, in order to prevent spoiling of the medication from exposure to air or other contaminants and to prevent leakage of the enclosure contents. The housings also open in directions opposite of the lancet device and opening for test media in order to prevent medication from contaminating blood glucose testing operation and from influencing test results. In alternative embodiments of the present invention, the enclosure is placed in positions on the back and sides of the apparatus.

Glucose tablets come in various sizes and the housing for hypoglycemia medication can be made in different sizes to accommodate variations. Concern is given to keeping the apparatus as compact as possible. The present invention can store enough glucose tablet medication for several treatments. Recommended treatment dose for glucose tablets is 3-4 tablets per hypoglycemic episode, but can vary depending on severity of hypoglycemia and patient intuition.

Another embodiment of the apparatus of the present invention is the ability to carry medication to treat hyperglycemia, often in the form of an insulin vial and syringe. In this embodiment the vial and syringe are stored in a vertical column or at the bottom of the apparatus, similar to the embodiment used for carrying hypoglycemia treatment medication. The vial can be stored in a location on the device different from the location of the syringe. Concern is given to the size of the apparatus of the present invention when it includes a treatment for hypoglycemia and hyperglycemia. Because treatment for hyperglycemia is not as urgent as that for hypoglycemia, preference is given to an apparatus with an enclosure for housing hypoglycemia medication prior to hyperglycemia. This preference is based on the constrained size and space requirements to maintain the present invention's ability to fit into a user's pocket on his or her clothing.

An additional embodiment for a cover or jacket to surround the outside of the device is presented in FIG. 7. The purpose of this jacket is to offer additional protection for the device against damage, contamination, or accidental operation, and to create an inconspicuous outer appearance for the apparatus. Alternative embodiments for this cover, or jacket are possible.

From the descriptions above, a number of advantages of some embodiments of the self-contained diabetic testing apparatus of the present invention become evident:
(a) There is no clutter of having to carry test meter, lancet, and test strip container separately.
(b) The apparatus is compact and can easily fit in a user's pants pocket.
(c) The apparatus can be manipulated quickly and easily, allowing for a predictable testing routine and fast execution of testing procedures.
(d) The unit is simple and discrete. It does not draw unwanted attention.
(e) There is a separate compartment for storage of used test media, allowing the media to be disposed of when convenient.
(f) The enclosure for housing test media can be detached and replaced to re-supply test media and replace dirty enclosures.
(g) Hypoglycemia or hyperglycemia medication is immediately available and accessible to the patient upon confirmation of blood glucose test result.

Accordingly, it will be understood that the self-contained blood glucose testing apparatus of the present invention can be used easily and conveniently and carried in a user's pocket, having tremendous impact on diabetics who must currently carry a purse, backpack, hip-pack, or other bag to transport their testing equipment in daily life.

Furthermore, it allows a diabetic patient the ability to carry the meter with him or herself at all times, so that they are not stuck in a dangerous situation where they need the ability to test blood sugar but did not bring the meter because they did not want to carry an extra bag. Common examples of such situations include, but are not limited to, exercising, walking, biking, going out with friends, shopping in a store and leaving the meter in the car, etc.

Additionally, because many test meters and test media are very sensitive to fluctuations in temperature, keeping the test meter with the user is the best way to avoid damage to the unit and avoid periods of inaccurate results or operation failure. When a meter is left behind in a locked car, the temperature can quickly rise or descend depending on weather and time of year. When the diabetic user returns to the car to use the device before operating the vehicle, they must act to heat or cool the device in order for it to work correctly or work at all. This usually involves waiting for some time and can create an uncomfortable or dangerous situation for the patient who is unable to accurately rely on his or her blood glucose meter or perform blood glucose analysis. The present invention will dramatically reduce a diabetic's exposure to unnecessary risk and stress, while simultaneously increasing their flexibility with the goal of providing them with as comfortable and normal of a life as possible.

In addition, the apparatus optimizes the flow of a blood glucose testing routine making the unavoidable task of testing blood glucose concentration quicker and simpler. Tedious manipulation of each component individually is greatly reduced by the invention.

The apparatus also aids in helping an individual who needs to check their blood sugar in a public setting to more readily conceal the fact that they have diabetes. Because the apparatus resembles a cellular phone or other small electronic device, the user appears to simply be checking the status of some common electronic gadget when using the meter, instead of performing a serious medical test involving lancing the skin with a sharp needle to produce an exposed blood sample. The flow and layout of the components for the apparatus also increase the speed at which a blood glucose test can be conducted, so that a user can finish the test in a few seconds and minimise his or her exposure to unwanted attention and intrusion by others.

Although the descriptions above contain many specificities, these should not be construed as limiting the scope of the embodiments, but as merely providing illustrations of some of the presently preferred embodiments. For example, the apparatus can have other shapes and sizes and the meter, lancet device, and storage compartment can be configured in different arrangements. Thus the scope of the embodiments should be determined by the appended claims and their legal equivalents, rather than by the examples given.

1. An apparatus for blood glucose testing comprising:
(a) a body having a front and a back,
(b) a meter unit,
(c) a lancet device comprising a trigger button for activating said lancet device, an arming slide for arming said lancet device, and a depth control mechanism for setting a lancet skin penetration depth
(d) an enclosure for housing new test media; and
(e) an enclosure for housing soiled test media.

2. The apparatus as claimed in claim 1, which further comprises an enclosure for housing medication to treat hypoglycemia and/or medication to treat hyperglycemia.

3. (canceled)

4. The apparatus as in claim 1 or 2, wherein one or both of said enclosures is a detachable cartridge.

5. The apparatus as in any of claims 1, 2, or 4, wherein said meter unit is positioned parallel and adjacent to said lancet device.

6. The apparatus as in any of claims 1,2, or 4, wherein said meter unit is positioned on top of said lancet device.

7. The apparatus as in any of claims 1-2 or 4-6, wherein said body further comprises a detachable cover for providing access to said lancet device for loading and unloading of lancets.

8. The apparatus as in any of claims 1-2 or 4-7, wherein said body further comprises a sliding cover for providing access to said lancet device for loading and unloading of lancets.

9. The apparatus as in any of claims 1-2 or 4-8, wherein said lancet device is detachable from said meter unit and enclosure.

10. The apparatus as in any of claim 2 or 4-9, wherein said enclosure for housing hypoglycemia and/or hyperglycemia treatment medication comprises a horizontal area.

11. The apparatus as in any of claim 2 or 4-9, wherein said enclosure for housing hypoglycemia and/or hyperglycemia treatment medication comprises a vertical column area.

12. The apparatus as in any of the preceding claims, further comprising a jacket to surround the outside of the apparatus.
